(19) United States
(12) Patent Application Publication
(10) Pub. No.: US 2025/0103796 A1
(43) Pub. Date: Mar. 27, 2025

(54) Expanding Textual Content Using Transfer Learning and Elicitive, Iterative Inference

(71) Applicant: Google LLC, Mountain View, CA (US)

(72) Inventors: Alvin Rajkomar, San Jose, CA (US); Eric Loreaux, San Francisco, CA (US); Yuchen Liu, Sunnyvale, CA (US); Ming-Jun Chen, Santa Clara, CA (US); Yi Zhang, Palo Alto, CA (US); Afroz Mohiuddin, Campbell, CA (US); Juraj Gottweis, Adliswil (CH)

(21) Appl. No.: 18/846,833

(22) PCT Filed: Feb. 23, 2023
(86) PCT No.: PCT/US2023/013707

§ 371 Date: Sep. 13, 2024

Related U.S. Application Data
(60) Provisional application No. 63/269,420, filed on Mar. 16, 2022.

(51) Int. Cl. G06F 40/166; G06V 30/41; G16H 10/60; G16H 70/20

(52) U.S. Cl. CPC G06F 40/166; G06V 30/41; G16H 10/60; G16H 70/20

(57) ABSTRACT

Example embodiments relate to expanding textual content using transfer learning and iterative inference. An example method includes receiving, by a computing device, a snippet of text that contains one or more terms expressed using succinct representations. The method also includes performing an iterative expansion, by the computing device, using the snippet of text as an input snippet of text. The iterative expansion includes receiving, by the computing device, the input snippet of text. The iterative expansion also includes determining, by the computing device using a machine-learned model, a set of intermediate expanded snippets. Each of the intermediate expanded snippets has an associated score based on the machine-learned model. Additionally, the iterative expansion includes: (i) repeating, by the computing device, the iterative expansion using a first intermediate expanded snippet or a second intermediate expanded snippet as the input snippet or (ii) outputting the input snippet as a final expanded snippet.

### REFERENCE TO RELATED APPLICATION

The present application claims priority to U.S. Provisional Application No. 63/269,420, filed Mar. 16, 2022, the contents of which are hereby incorporated by reference.

### BACKGROUND

Unless otherwise indicated herein, the materials described in this section are not prior art to the claims in this application and are not admitted to be prior art by inclusion in this section.

Clinicians write medical notes to record and communicate diagnoses and treatments, but the language they use (sometimes referred to as “medicalese”) may be unfamiliar to patients and even doctors from other specialties. Medicalese is characterized by shorthand and abbreviations that may be medical jargon (e.g., “hit” for “heparin induced thrombocytopenia”), ambiguous terms made clear only with expertise and context (“ms” for “multiple sclerosis” in some contexts or “mental status” in other contexts), or clinical vernacular (“cb” for “complicated by”). As such, understanding medicalese may be challenging.

Additionally, machine learning is a field in computing that involves a computing device training a model using “training data.” There are two primary classifications of methods of training models: supervised learning and unsupervised learning. In supervised learning, the training data is classified into data types, and the model is trained to look for variations/similarities among known classifications. In unsupervised learning, the model is trained using training data that is unclassified. Thus, in unsupervised learning, the model is trained to identify similarities based on unlabeled training data.

Once the model has been trained on the training data, the model can then be used to analyze new data (sometimes called “test data”). Based on the model's training, a computing device can use the trained model to evaluate the similarity of the test data.

There are numerous types of machine-learned models, each having its own set of advantages and disadvantages. One popular machine-learned model is an artificial neural network. The artificial neural network involves layers of structure, each trained to identify certain features of an input (e.g., an input image, an input sound file, or an input text file). Each layer may be built upon sub-layers that are trained to identify sub-features of a given feature. For example, an artificial neural network may identify composite objects within an image based on sub-features such as edges or textures.

Given the current state of computing power, in some artificial neural networks many such sub-layers can be established during training of a model. Artificial neural networks that include multiple sub-layers are sometimes referred to as “deep neural networks.” In some deep neural networks, there may be hidden layers and/or hidden sub-layers that identify composites or superpositions of inputs. Such composites or superpositions may not be human-interpretable.

### SUMMARY

This disclosure relates to expanding textual content using transfer learning and iterative inference. In many contexts (e.g., in some highly technical contexts, such as a medical context), succinct representations such as abbreviations, acronyms, and/or shorthands may be used to communicate long-form information in a more condensed manner. For example, a clinician may write a medical note (e.g., in what is sometimes referred to as “medicalese”) in order to communicate diagnoses and/or treatment regimens. Such medical notes may contain a series of succinct representations. In order to make the succinct representations understandable (e.g., by other trained professionals, such as other trained physicians, and/or by laypeople, such as patients) and/or in order to more easily compare one set of text to another (e.g., particularly in contexts where different people may use different succinct representations for the same term or phrase), example embodiments herein utilize two machine-learning techniques to expand the succinct representations into long-form. First, example embodiments may include a technique for training a machine-learned model used to perform the expansion. The machine-learned model may be trained using snippets of publicly available website data. Then, via transfer learning, this machine-learned model may be used to expand the succinct representations. Second, example embodiments may include performing an elicitive, iterative inference using the machine-learned model (e.g., the machine-learned model trained using publicly available website data or another machine-learned model) by performing a beam search using the machine-learned model and determining which of multiple possible results should be used for iteration.

In one aspect, a method is provided. The method includes receiving, by a computing device, a snippet of text that contains one or more terms expressed using succinct representations. The method also includes performing an iterative expansion, by the computing device, using the snippet of text as an input snippet of text. The iterative expansion includes receiving, by the computing device, the input snippet of text. The iterative expansion also includes determining, by the computing device using a machine-learned model, a set of intermediate expanded snippets. Each of the intermediate expanded snippets has an associated score based on the machine-learned model. A first intermediate expanded snippet corresponds to a highest associated score. A second intermediate expanded snippet corresponds to a second highest associated score. Additionally, the iterative expansion includes, if the first intermediate expanded snippet is different from the input snippet of text, repeating, by the computing device, the iterative expansion using the first intermediate expanded snippet as the input snippet. Further, the iterative expansion includes, if the first intermediate expanded snippet is the same as the input snippet of text and the second highest associated score is greater than or equal to a threshold score, repeating, by the computing device, the iterative expansion using the second intermediate expanded snippet as the input snippet. In addition, the iterative expansion includes, if the first intermediate expanded snippet is the same as the input snippet of text and the second highest associated score is less than the threshold score, outputting the input snippet as a final expanded snippet.

In another aspect, a method is provided. The method includes parsing, using a plurality of computing devices, webpages to obtain a plurality of training snippets of text. The method also includes separating, by the plurality of computing devices, the plurality of training snippets into a plurality of training groups. Each of the training groups comprises one or more of the training snippets. Each of the training groups is assigned to a subset of the plurality of computing devices. Additionally, the method includes determining, for each respective training group by the respective subset of the plurality of computing devices, a plurality of inclusion values. Each of the inclusion values is based on a number of times a respective expanded representation of a term appears within the respective training group. Further, the method includes determining, by the plurality of computing devices for each training snippet, whether to include the respective training snippet in a training set based on the term that has the largest inclusion value in the respective training snippet. In addition, the method includes replacing, by the plurality of computing devices with a reverse substitution probability, for each term having an expanded representation in each training snippet included in the training set, the respective expanded representation with a succinct representation of the respective term. Still further, the method includes outputting the training set.

In an additional aspect, a non-transitory, computer-readable medium having instructions stored therein is provided. The instructions, when executed by a processor, perform a method. The method includes receiving a snippet of text that contains one or more terms expressed using succinct representations. The method also includes performing an iterative expansion using the snippet of text as an input snippet of text. The iterative expansion includes receiving the input snippet of text. The iterative expansion also includes determining, using a machine-learned model, a set of intermediate expanded snippets. Each of the intermediate expanded snippets has an associated score based on the machine-learned model. A first intermediate expanded snippet corresponds to a highest associated score. A second intermediate expanded snippet corresponds to a second highest associated score. Additionally, the iterative expansion includes, if the first intermediate expanded snippet is different from the input snippet of text and the highest associated score is greater than a threshold score, repeating the iterative expansion using the first intermediate expanded snippet as the input snippet. Further, the iterative expansion includes, if the first intermediate expanded snippet is the same as the input snippet of text and the second highest associated score is greater than the threshold score, repeating the iterative expansion using the second intermediate expanded snippet as the input snippet. In addition, the iterative expansion includes, if the first intermediate expanded snippet is the same as the input snippet of text and the second highest associated score is less than the threshold score, outputting the input snippet as a final expanded snippet.

These as well as other aspects, advantages, and alternatives will become apparent to those of ordinary skill in the art by reading the following detailed description, with reference, where appropriate, to the accompanying drawings.

### BRIEF DESCRIPTION OF THE DRAWINGS

FIG. 1 is a diagram illustrating training and inference phases of a machine-learning model, according to example embodiments.

FIG. 2 is a simplified block diagram showing some of the components of a computing device, according to example embodiments.

FIG. 3 is an illustration of a method of generating a training set, according to example embodiments.

FIG. 4 is an illustration of a method of expanding text within a clinical note, according to example embodiments.

FIG. 5A is an illustration of an iterative, elicitive inference method, according to example embodiments.

FIG. 5B is an illustration of an iterative, elicitive inference method, according to example embodiments.

FIG. 6A illustrates experimental results, according to example embodiments.

FIG. 6B illustrates experimental results, according to example embodiments.

FIG. 6C illustrates experimental results, according to example embodiments.

FIG. 6D illustrates experimental results, according to example embodiments.

FIG. 7A is a flowchart illustration of a method, according to example embodiments.

FIG. 7B is a flowchart illustration of a method, according to example embodiments.

FIG. 8 is a flowchart illustration of a method, according to example embodiments.

### DETAILED DESCRIPTION

Example methods and systems are contemplated herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. Further, some of the illustrated elements may be combined or omitted. Yet further, an example embodiment may include elements that are not illustrated in the figures.

The terms “short form,” “abbreviation,” “acronym,” “succinct representation,” etc. are used throughout this disclosure. It is understood that the term “succinct representation,” is meant to represent a genus, of which “abbreviation,” “acronym,” etc. are species. In other words, a “succinct representation” (i.e., a “short form”) is a shortened/shorter form of a given word or set of words that facilitates fewer characters used to convey the phrase in question. Further, a “succinct representation” may be a shortened/short representation of a corresponding “expanded form” of the same word/set of words. Examples of such shortened forms may include abbreviations (e.g., “pt” is an abbreviation for “patient”) and acronyms (e.g., “ms” is an acronym for “multiple sclerosis”). While certain types of succinct representations may be used throughout the disclosure, it is understood that in many cases other types of succinct representations may be equally valid, depending on context.

The terms “expanded form,” “long form,” “extended form,” etc. are likewise used throughout the disclosure. These terms are understood to be interchangeable, depending on context. For example, each of these terms may denote a longer form (e.g., a full-length form) of a given word or set of words that may use more characters to convey the phrase in question than a corresponding succinct representation. The expanded form has the same meaning (semantic content) as the succinct representation (e.g., it would be interpreted as having the same information content by someone familiar with the succinct representation).

With respect to embodiments that include making determinations using a machine learning model, interactions by the computing device with cloud-based servers, or otherwise involve sharing captured images or text with other computing devices, a user may be provided with controls allowing the user to make an election as to both if and when systems, programs, or features described herein may enable collection of user information (e.g., information about a user's social network, social actions, activities, profession, health, a user's preferences, or a user's current location), and if the user is sent content or communications from a server. In addition, certain data may be treated in one or more ways before it is stored or used, so that personally identifiable information is redacted, de-identified, or removed. For example, a user's identity may be treated so that no personally identifiable information can be determined for the user; a user's geographic location may be generalized where location information is obtained (such as to a city, ZIP code, or state level), so that a particular location of a user cannot be determined; and/or a user's health-related information (e.g., sex, age, weight, height, etc.) may be excised, so that a particular user's condition(s) may not be associated with the user. Thus, the user may have control over what information is collected about the user, how that information is used, and/or what information is provided to the user and/or to other users.

### I. Overview

The expansion of medical abbreviations is a hard natural-language-understanding (NLU) problem because abbreviations may be ambiguous and/or domain specific (e.g., “AF” could refer to “atrial fibrillation” or “afebrile” depending on the context). In related natural language systems, such as those that translate text from one language to another (e.g., using machine learning), large corpuses of original and translated text exist. For the medical domain, however, no pre-existing corpus of medicalese and more-understandable translations exist, and the words/concepts in the medical text are unique to the domain. Further, because of privacy concerns surrounding sensitive information, there are not large amounts of training data that are publicly available.

Example embodiments described herein include techniques for training a machine-learned model that can be usable to expand succinct representations (e.g., abbreviations). Such succinct representations may be diagnosis/treatment abbreviations used in the medical context (e.g., in a clinical note prepared by an attending physician). Thus, the succinct representations may represent (e.g., encode) real-world medical data describing the medical condition of corresponding subjects (e.g., human subjects). This real world medical data may be measurement data derived by making measurements of the subjects using sensors (e.g., thermometers, imaging devices, and/or apparatuses used to process in vitro samples of bodily fluids) and/or criterion data indicating that at least one value of measurement data obtained by measuring a subject obeys a criterion, such as criterion data indicating that a measured value is outside an expected range (e.g., a normal range for human subjects), such as “elevated temperature.” Additionally or alternatively, the medical data may encode diagnostic judgements data generated by a human or automated diagnostic system based on the measurement data. However, example embodiments described herein are not limited to the medical context. For example, internal code names within a corporation or accounting terms used within a finance setting may also be expanded using the machine-learned model. Training the machine-learned model according to techniques described herein may include generating a training dataset.

In order to generate the training data, example embodiments may include taking publicly available website data (e.g., publicly available data relating to a given area of interest, such as medicine), splitting that website data into snippets (e.g., segments between one sentence and three sentences in length), and distributing those snippets among a number of computing clusters (i.e., “shards”). These computing clusters may then perform reverse substitution (e.g., replacing long-form representations with succinct representations) on one or more snippets from the publicly available website data in order to generate the training data. Additionally, in order to generate the training dataset, example embodiments may include determining whether to include a given snippet in the training dataset based on an inclusion value (e.g., an inclusion value that, when compared to other inclusion values within a given shard, serves to upsample some of the less-frequent long-form representations (i.e. increase their occurrence frequency in the training dataset) and downsample some of the more-frequent long-form representations (i.e. decrease their occurrence frequency in the training dataset). Further, in order to generate the training data, example embodiments may include determining whether to replace the snippets for inclusion in the training data with succinct representations (e.g., via reverse substitution) based on a reverse substitution probability. For example, for each long-form representation, there may be a corresponding reverse substitution probability value. Further, a given long-form representation may be substituted with a corresponding succinct representation with a probability given by the reverse substitution probability value.

Additionally, example embodiments described herein also include techniques for performing inferences using a machine-learned model (e.g., once the model has been trained). Such inference techniques may be referred to herein as “elicitive, iterative inference,” “iterative, elicitive inference,” “iterative and elicitive inference,” “elicitive and iterative inference,” or simply “elicitive inference.” In typical iterative-only inference techniques, the result of an inference step is fed back into the machine-learned model as an input for a future inference step repeatedly until the result of applying the machine-learned model in a given inference step matches the input in that inference step. Once the result matches the input, the input for that given step is determined to be the ultimate output of the inference process.

In example embodiments herein, though, elicitive inference may instead be performed. Elicitive inference, unlike traditional iterative inference, may include generating a series of potential results for each inference step (e.g., using a beam search). Each of the potential results may also have an associated score (e.g., a probability of the potential result being the correct result based on the input, where the probability may be based on the training performed to train the machine-learned model). Next in elicitive inference, the potential result with the highest score is compared to the input for that step of the inference process. If the potential result with the highest score is different from the input for that step of the inference process, an additional inference step is performed with the potential result having the highest score being used as the input for the additional inference step. If the potential result with the highest score is the same as the input for that step of the inference process, though, the potential result with the second highest score is reviewed. If the second highest score is greater than or equal to a threshold score, an additional inference step is performed with the potential result having the second highest score being used as the input for the additional inference step. If the second highest score is less than the threshold score, though, the input for that step of the inference process is output as the ultimate output of the inference process. Using this elicitive inference technique, the accuracy of the results generated using the machine-learned model can be further augmented.

### II. Example Systems

The following description and accompanying drawings will elucidate features of various example embodiments. The embodiments provided are by way of example, and are not intended to be limiting. As such, the dimensions of the drawings are not necessarily to scale.

A machine-learned model as described herein may include, but is not limited to: an artificial neural network (e.g., a convolutional neural network, a recurrent neural network, a Bayesian network, a hidden Markov model, a Markov decision process, a logistic regression function, a suitable statistical machine-learning algorithm, and/or a heuristic machine-learning system), a support vector machine, a regression tree, an ensemble of regression trees (also referred to as a regression forest), a decision tree, an ensemble of decision trees (also referred to as a decision forest), or some other machine-learning model architecture or combination of architectures.

An artificial neural network (ANN) could be configured in a variety of ways. For example, the ANN could include two or more layers, could include units having linear, logarithmic, or otherwise-specified output functions, could include fully or otherwise-connected neurons, could include recurrent and/or feed-forward connections between neurons in different layers, could include filters or other elements to process input information and/or information passing between layers, or could be configured in some other way to facilitate the generation of predicted color palettes based on input images.

An ANN could include one or more filters that could be applied to the input, and the outputs of such filters could then be applied to the inputs of one or more neurons of the ANN. For example, such an ANN could be or could include a convolutional neural network (CNN). Convolutional neural networks are a variety of ANNs that are configured to facilitate ANN-based classification or other processing based on images or other large-dimensional inputs whose elements are organized within two or more dimensions. The organization of the ANN along these dimensions may be related to some structure in the input structure (e.g., as relative location within the two-dimensional space of an image can be related to similarity between pixels of the image).

In example embodiments, a CNN includes at least one two-dimensional (or higher-dimensional) filter that is applied to an input; the filtered input is then applied to neurons of the CNN (e.g., of a convolutional layer of the CNN). The convolution of such a filter and an input could represent the color values of a pixel or a group of pixels from the input, in embodiments where the input is an image. A set of neurons of a CNN could receive respective inputs that are determined by applying the same filter to an input. Additionally or alternatively, a set of neurons of a CNN could be associated with respective different filters and could receive respective inputs that are determined by applying the respective filter to the input. Such filters could be trained during training of the CNN or could be pre-specified. For example, such filters could represent wavelet filters, center-surround filters, biologically-inspired filter kernels (e.g., from studies of animal visual processing receptive fields), or some other pre-specified filter patterns.

A CNN or other variety of ANN could include multiple convolutional layers (e.g., corresponding to respective different filters and/or features), pooling layers, rectification layers, fully connected layers, or other types of layers. Convolutional layers of a CNN represent convolution of an input image, or of some other input (e.g., of a filtered, downsampled, or otherwise-processed version of an input image), with a filter. Pooling layers of a CNN apply non-linear downsampling to higher layers of the CNN, e.g., by applying a maximum, average, L2-norm, or other pooling function to a subset of neurons, outputs, or other features of the higher layer(s) of the CNN. Rectification layers of a CNN apply a rectifying nonlinear function (e.g., a non-saturating activation function, a sigmoid function) to outputs of a higher layer. Fully connected layers of a CNN receive inputs from many or all of the neurons in one or more higher layers of the CNN. The outputs of neurons of one or more fully connected layers (e.g., a final layer of an ANN or CNN) could be used to determine information about areas of an input image (e.g., for each of the pixels of an input image) or for the image as a whole.

Neurons in a CNN can be organized according to corresponding dimensions of the input. For example, where the input is an image (a two-dimensional input, or a three-dimensional input where the color channels of the image are arranged along a third dimension), neurons of the CNN (e.g., of an input layer of the CNN, of a pooling layer of the CNN) could correspond to locations in the two-dimensional input image. Connections between neurons and/or filters in different layers of the CNN could be related to such locations. For example, a neuron in a convolutional layer of the CNN could receive an input that is based on a convolution of a filter with a portion of the input image, or with a portion of some other layer of the CNN, that is at a location proximate to the location of the convolutional-layer neuron. In another example, a neuron in a pooling layer of the CNN could receive inputs from neurons, in a layer higher than the pooling layer (e.g., in a convolutional layer, in a higher pooling layer), that have locations that are proximate to the location of the pooling-layer neuron.

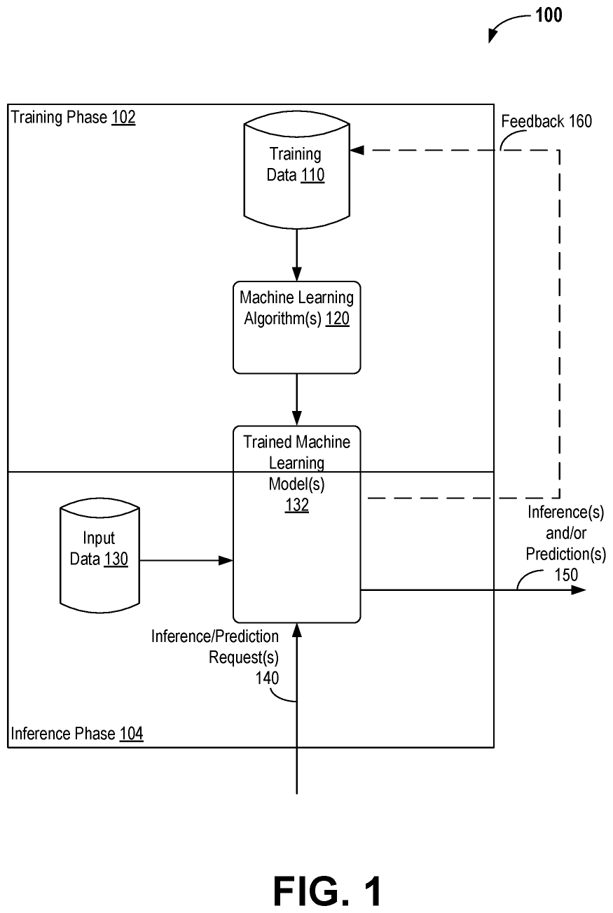

FIG. 1 shows diagram 100 illustrating a training phase 102 and an inference phase 104 of trained machine-learning model(s) 132, in accordance with example embodiments. Some machine-learning techniques involve training one or more machine-learning algorithms, on an input set of training data to recognize patterns in the training data and provide output inferences and/or predictions about (patterns in the) training data. Such output could take the form of filtered or otherwise modified versions of the input (e.g., an input image could be modified by the machine-learning model to appear as though foreground content is in-focus while background content is out of focus). The resulting trained machine-learning algorithm can be termed as a trained machine-learning model or, simply, a machine-learned model. For example, FIG. 1 shows training phase 102 where one or more machine-learning algorithms 120 are being trained on training data 110 to become trained machine-learning model 132. Then, during inference phase 104, trained machine-learning model 132 can receive input data 130 and one or more inference/prediction requests 140 (e.g., as part of input data 130) and responsively provide as an output one or more inferences and/or predictions 150.

As such, trained machine-learning model(s) 132 can include one or more models of one or more machine-learning algorithms 120. Machine-learning algorithm(s) 120 may include, but are not limited to: an artificial neural network (e.g., a herein-described convolutional neural networks, a recurrent neural network, a Bayesian network, a hidden Markov model, a Markov decision process, a logistic regression function, a suitable statistical machine-learning algorithm, and/or a heuristic machine-learning system), a support vector machine, a regression tree, an ensemble of regression trees (also referred to as a regression forest), a decision tree, an ensemble of decision trees (also referred to as a decision forest), or some other machine-learning model architecture or combination of architectures. Machine-learning algorithm(s) 120 may be supervised or unsupervised, and may implement any suitable combination of online and offline learning.

In some examples, machine-learning algorithm(s) 120 and/or trained machine-learning model(s) 132 can be accelerated using on-device coprocessors, such as graphic processing units (GPUs), tensor processing units (TPUs), digital signal processors (DSPs), and/or application specific integrated circuits (ASICs). Such on-device coprocessors can be used to speed up machine-learning algorithm(s) 120 and/or trained machine-learning model(s) 132. In some examples, trained machine-learning model(s) 132 can execute to provide inferences on, be trained on, and/or reside on a particular computing device, and/or otherwise can make inferences for the particular computing device.

During training phase 102, machine-learning algorithm(s) 120 can be trained by providing at least training data 110 as training input using unsupervised, supervised, semi- supervised, and/or reinforcement learning techniques. Unsupervised learning involves providing a portion (or all) of training data 110 to machine-learning algorithm(s) 120 and machine-learning algorithm(s) 120 determining one or more output inferences based on the provided portion (or all) of training data 110. Supervised learning involves providing a portion of training data 110 to machine-learning algorithm(s) 120, with machine-learning algorithm(s) 120 determining one or more output inferences based on the provided portion of training data 110, and the output inference(s) are either accepted or corrected based on correct results associated with training data 110. In some examples, supervised learning of machine-learning algorithm(s) 120 can be governed by a set of rules and/or a set of labels for the training input, and the set of rules and/or set of labels may be used to correct inferences of machine-learning algorithm(s) 120. Items of the training data 110 may include, as training inputs for the machine learning models 132, corresponding snippets of text that include one or more terms expressed using succinct representations. The text in the training inputs may be text obtained by character recognition from an image of a (e.g., handwritten) clinical note written by a human (e.g. a doctor) to describe the medical condition of a specific patient. Alternatively, a given training input may include an image of the clinical note. In some embodiments, the training inputs for the machine learning models 132 may further include non-textual forms of data, such as measurement data obtained from human subjects using sensors (e.g., in the form of corresponding images and/or numerical measurement data). At least some items of the training data may further include corresponding correct results (e.g., inferences), such as corresponding expanded snippets of text having the same meaning as the text in the training input of the corresponding item of training data and in which one or more of the succinct representations are replaced by long-form representations.

Semi-supervised learning involves having correct results for part, but not all, of training data 110. During semi-supervised learning, supervised learning is used for a portion of training data 110 having correct results, and unsupervised learning is used for a portion of training data 110 not having correct results. Reinforcement learning involves machine-learning algorithm(s) 120 receiving a reward signal regarding a prior inference, where the reward signal can be a numerical value. During reinforcement learning, machine-learning algorithm(s) 120 can output an inference and receive a reward signal in response, where machine-learning algorithm(s) 120 are configured to try to maximize the numerical value of the reward signal. In some examples, reinforcement learning also utilizes a value function that provides a numerical value representing an expected total of the numerical values provided by the reward signal over time. In some examples, machine-learning algorithm(s) 120 and/or trained machine-learning model(s) 132 can be trained using other machine-learning techniques, including but not limited to, incremental learning and curriculum learning.

In some examples, machine-learning algorithm(s) 120 and/or trained machine- learning model(s) 132 can use transfer-learning techniques. For example, transfer-learning techniques can involve trained machine-learning model(s) 132 being pre-trained on one set of data and additionally trained using training data 110. More particularly, machine-learning algorithm(s) 120 can be pre-trained on data from one or more computing devices and a resulting trained machine-learning model provided to computing device CD1, where CD1 is intended to execute the trained machine-learning model during inference phase 104. Then, during training phase 102, the pre-trained machine-learning model can be additionally trained using training data 110, where training data 110 can be derived from kernel and non-kernel data of computing device CD1. This further training of the machine-learning algorithm(s) 120 and/or the pre-trained machine-learning model using training data 110 of CD1's data can be performed using either supervised or unsupervised learning. Once machine-learning algorithm(s) 120 and/or the pre-trained machine-learning model has been trained on at least training data 110, training phase 102 can be completed. The trained resulting machine-learning model can be utilized as at least one of trained machine-learning model(s) 132.

In particular, once training phase 102 has been completed, trained machine-learning model(s) 132 can be provided to a computing device, if not already on the computing device. Inference phase 104 can begin after trained machine-learning model(s) 132 are provided to computing device CD1.

During inference phase 104, trained machine-learning model(s) 132 can receive input data 130 and generate and output one or more corresponding inferences and/or predictions 150 about input data 130. As such, input data 130 can be used as an input to trained machine-learning model(s) 132 for providing corresponding inference(s) and/or prediction(s) 150 to kernel components and non-kernel components. For example, trained machine-learning model(s) 132 can generate inference(s) and/or prediction(s) 150 in response to one or more inference/prediction requests 140. In some examples, trained machine-learning model(s) 132 can be executed by a portion of other software. For example, trained machine-learning model(s) 132 can be executed by an inference or prediction daemon to be readily available to provide inferences and/or predictions upon request. Input data 130 can include data from computing device CD1 executing trained machine-learning model(s) 132 and/or input data from one or more computing devices other than CD1.

Items of input data 130 can include a corresponding snippet of text that contains one or more terms expressed using succinct representations. Alternatively or additionally, input data 130 can include a collection of images provided by one or more sources. The collection of images can include video frames, images resident on computing device CD1, and/or other images. Other types of input data are possible as well.

Inference(s) and/or prediction(s) 150 can include a corresponding expanded snippet of text having the same meaning as the snippet of text of the corresponding items of input data 130 and that contains one or more long-form terms having a meaning equivalent to the succinct representations of the corresponding items of input data 130. Alternatively or additionally, the inference(s) and/or predictions 150 may include output images, output intermediate images, numerical values, and/or other output data produced by trained machine-learning model(s) 132 operating on input data 130 (and training data 110). In some examples, trained machine-learning model(s) 132 can use output inference(s) and/or prediction(s) 150 as input feedback 160. Trained machine-learning model(s) 132 can also rely on past inferences as inputs for generating new inferences.

A conditioned, axial self-attention based neural network can be an example of machine-learning algorithm(s) 120. After training, the trained version of the neural network can be an example of trained machine-learning model(s) 132. In this approach, an example of inference/prediction request(s) 140 can be a request to predict one or more colorizations of a grayscale image and a corresponding example of inferences and/or prediction(s) 150 can be an output image including the one or more colorizations of the grayscale image.

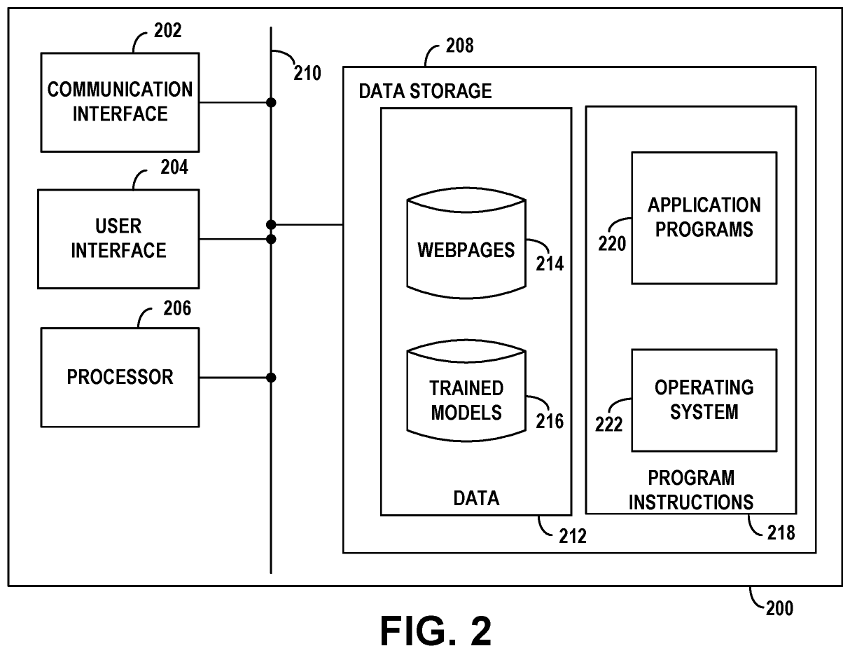

FIG. 2 illustrates an example computing device 200 that may be used to implement the methods described herein. By way of example and without limitation, computing device 200 may be a cellular mobile telephone (e.g., a smartphone), a computer (such as a desktop, notebook, tablet, or handheld computer, a server), elements of a cloud computing system, a robot, a drone, an autonomous vehicle, or some other type of device. It should be understood that computing device 200 may represent a physical computing device such as a server, a particular physical hardware platform on which a machine-learning application operates in software, or other combinations of hardware and software that are configured to carry out machine-learning functions as described herein.

As shown in FIG. 2, computing device 200 may include a communication interface 202, a user interface 204, a processor 206, and data storage 208, all of which may be communicatively linked together by a system bus, network, or other connection mechanism 210.

Communication interface 202 may function to allow computing device 200 to communicate, using analog or digital modulation of electric, magnetic, electromagnetic, optical, or other signals, with other devices, access networks, and/or transport networks. Thus, communication interface 202 may facilitate circuit-switched and/or packet-switched communication, such as plain old telephone service (POTS) communication and/or Internet protocol (IP) or other packetized communication. For instance, communication interface 202 may include a chipset and antenna arranged for wireless communication with a radio access network or an access point. Also, communication interface 202 may take the form of or include a wireline interface, such as an Ethernet, Universal Serial Bus (USB), or High-Definition Multimedia Interface (HDMI) port. Communication interface 202 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 202. Furthermore, communication interface 202 may include multiple physical communication interfaces (e.g., a Wifi interface, a BLUETOOTH® interface, and a wide-area wireless interface).

In some embodiments, communication interface 202 may function to allow computing device 200 to communicate with other devices, remote servers, access networks, and/or transport networks. For example, the communication interface 202 may function to access one or more machine-learning models and/or input therefor via communication with a remote server or other remote device or system in order to allow the computing device 200 to use the machine-learned model to generate outputs (e.g., class values for inputs, filtered or otherwise modified versions of image inputs) based on input data. For example, the computing device 200 could be an image server and the remote system could be a smartphone containing an image to be applied to a machine-learning model.

User interface 204 may function to allow computing device 200 to interact with a user, for example to receive input from and/or to provide output to the user. Thus, user interface 204 may include input components such as a keypad, keyboard, touch-sensitive or presence-sensitive panel, computer mouse, trackball, joystick, microphone, and so on. User interface 204 may also include one or more output components such as a display screen which, for example, may be combined with a presence-sensitive panel. The display screen may be based on cathode-ray tube (CRT), liquid-crystal display (LCD), light-emitting diode (LED) technologies, and/or other technologies now known or later developed. User interface 204 may also be configured to generate audible output(s), via a speaker, speaker jack, audio output port, audio output device, earphones, and/or other similar devices.

Processor 206 may include one or more general purpose processors (e.g., microprocessors) and/or one or more special purpose processors (e.g., DSPs, GPUs, floating point units (FPUs), network processors, TPUs, or ASICs). In some instances, special purpose processors may be capable of image processing, image alignment, merging images, executing artificial neural networks, or executing convolutional neural networks, among other applications or functions. Data storage 208 may include one or more volatile and/or non- volatile storage components, such as magnetic, optical, flash, or organic storage, and may be integrated in whole or in part with processor 206. Data storage 208 may include removable and/or non-removable components.

Processor 206 may be capable of executing program instructions 218 (e.g., compiled or non-compiled program logic and/or machine code) stored in data storage 208 to carry out the various functions described herein. Therefore, data storage 208 may include a non-transitory, computer-readable medium, having stored thereon program instructions that, upon execution by computing device 200, cause computing device 200 to carry out any of the methods, processes, or functions disclosed in this specification and/or the accompanying drawings. The execution of program instructions 218 by processor 206 may result in processor 206 using data 212.

By way of example, program instructions 218 may include an operating system 222 (e.g., an operating system kernel, device driver(s), and/or other modules) and one or more application programs 220 (e.g., functions for executing trained machine-learning models) installed on computing device 200. Data 212 may include input web data 214 and/or one or more trained machine-learning models 216. Webpages 214 (e.g., stored as portable document format (PDF) files or hypertext markup language (HTML) files) may be used to train machine-learning models and/or to generate some other model output as described herein.

Application programs 220 may communicate with operating system 222 through one or more application programming interfaces (APIs). These APIs may facilitate, for instance, application programs 220 reading and/or writing a trained machine-learning model 216, transmitting or receiving information via communication interface 202, receiving and/or displaying information on user interface 204, and so on.

Application programs 220 may take the form of “apps” that could be downloadable to computing device 200 through one or more online application stores or application markets (via, e.g., the communication interface 202). However, application programs can also be installed on computing device 200 in other ways, such as via a web browser or through a physical interface (e.g., a USB port) of the computing device 200.

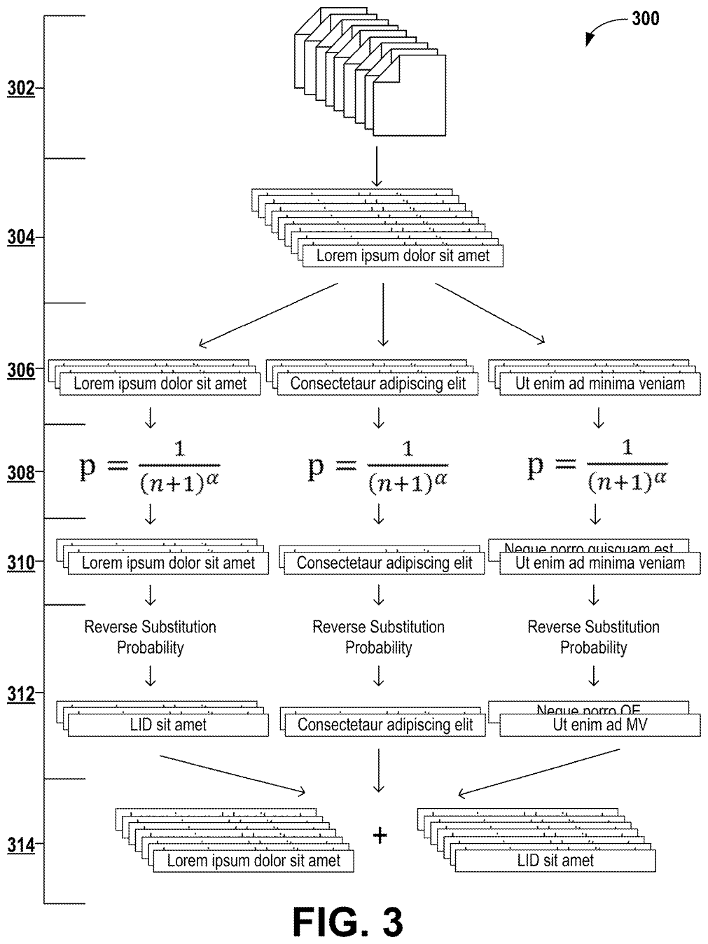

FIG. 3 is an illustration of a method 300 of generating a training set (e.g., usable to train a machine-learned model), according to example embodiments. The training set may, for example, be used as the training data 110 in diagram 100 of FIG. 1. The training set generated may include a set of long-forms/expanded forms and corresponding succinct representations thereof. As described herein, the method 300 may include performing reverse substitution on expanded forms based on predetermined corresponding succinct representations (e.g., according to a predefined dictionary).

At a first step 302 of the method 300, publicly available web data (e.g., a series of webpages and/or websites) may be retrieved. The publicly available web data may be stored within a non-transitory, computer-readable medium (e.g., a hard drive), a series of non- transitory, computer-readable media (e.g., multiple hard drives), within a random-access memory (RAM), and/or within cloud storage. Further, the publicly available web data may be stored in a variety of formats (e.g., one or more portable document format (PDF) files, one or more hypertext markup language (HTML) files, one or more extensible markup language (XML) files, etc.). Additionally or alternatively, the publicly available web data retrieved may represent information corresponding to a specific discipline. For example, the publicly available web data retrieved may only correspond to health-related web data or medical web data (e.g., medical information used for public education). The publicly available web data may be retrieved by one or more web crawlers executed by one or more computing devices, for example.

There may be multiple benefits to using publicly available web data for training the machine-learned model. First, there exists a large amount of publicly available web data relating to any given subject matter. As such, the training set generated using the publicly available web data may be rather large, leading to a robust machine-learned model. Additionally, unlike private data (e.g., taken from actual clinical notes or discharge summaries relating to diagnoses/treatments of patients), publicly available web data may contain substantially less or no personal information (e.g., no private medical information). As such, protections of personal privacy can be maintained while still adequately training the machine-learned model (e.g., and without the need to apply a scrubbing process to remove personal information).

At step 304, upon retrieving the publicly available web data, the method 300 may include breaking the publicly available web data into snippets of text. In various embodiments, snippets of text may have various lengths. For example, a snippet of text may include a single word, a set of consecutive words (e.g., two words in length, three words in length, four words in length, etc.), a single sentence, a set of consecutive sentences (e.g., two sentences in length, three sentences in length, four sentences in length, etc.), a single paragraph, a set of consecutive paragraphs (e.g., two paragraphs in length, three paragraphs in length, four paragraphs in length, etc.), etc. Further, in some embodiments, each of the snippets of text may be the same length. Alternatively, the snippets of text may be of variable length (e.g., depending on the context of the surrounding text). In some embodiments, for example, snippets may be between one sentence and three sentences in length.

At step 306, upon dividing publicly available web data into different snippets, the snippets may be separated into a plurality of training groups. The training groups may then be distributed to different computing devices (e.g., different computing devices within a given computing cluster or distributed computing devices located in different remote locations) and/or sets of computing devices (e.g., different server farms). The various computing devices or sets of computing devices may be referred to as “shards” herein. For example, in some embodiments, the snippets may be separated into tens, hundreds, thousands, tens of thousands, hundreds of thousands, millions, etc. of training groups that are subsequently distributed to computing devices. For illustration purposes, three shards are represented in FIG. 3. In some embodiments, the number of snippets put into a given training group that is subsequently assigned to a given computing device may be determined based on the available computational resources for the given computing device. In some embodiments, rather than retrieving all the publicly available web data, parsing that web data to obtain snippets of text, separating those snippets into training groups, and then distributing the training groups, each of the computing devices may independently retrieve publicly available web data (e.g., perhaps in communication with one another to prevent the same publicly available web data being incorporated into different training groups/processed repeatedly by different computing devices) and parse that web data into snippets for processing. In other words, steps 302, 304, and 306 may all be performed on separate computing devices.

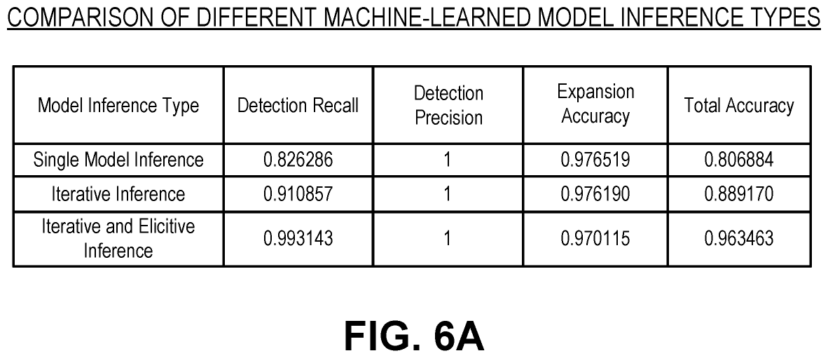

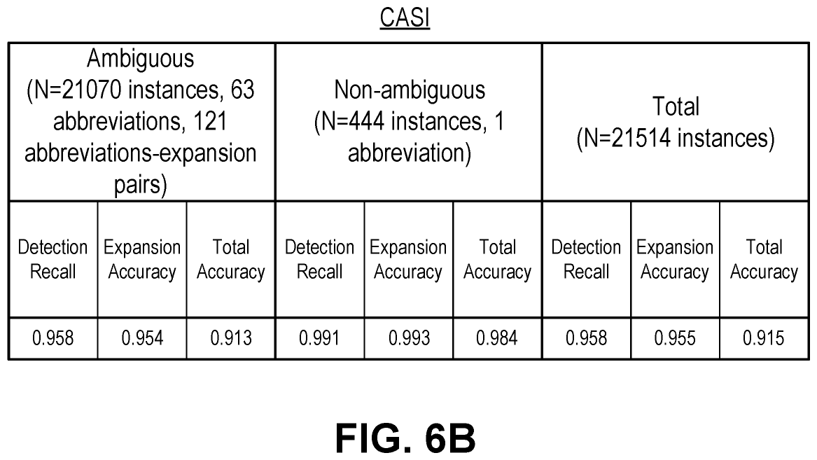

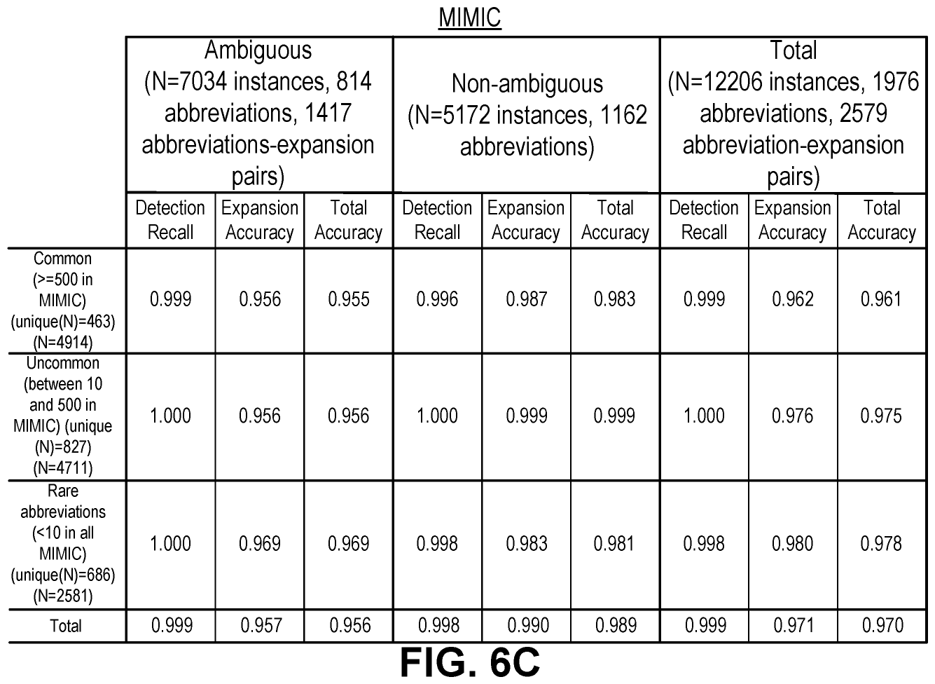

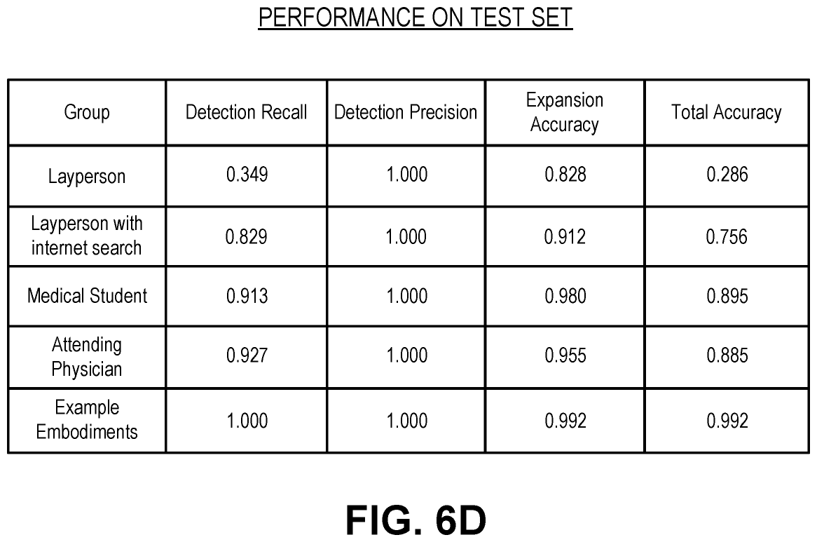

At step 308, upon receiving the training group that includes the snippets of text, the method 300 may include each of the shards determining inclusion values for each of the expanded forms contained within at least one snippet within the respective training group assigned to that computing device/set of computing devices. For example, the computing device(s) within a given shard may each be provided with one or more dictionaries. The dictionary may include pairs (e.g., 5,000 or more unique pairs) of succinct representations and their associated expanded forms (e.g., the dictionary includes “AF: atrial fibrillation,” “AF: afebrile,” “afib: atrial fibrillation,” “as: aortic stenosis,” “cdgs: carbohydrate deficient glycoprotein syndrome,” “pt: patient,” “us: ultrasound,” etc.). Such a dictionary may have been generated (e.g., by one or more of the computing devices spread across the shards or another computing device) based on one or more dictionaries available in the given discipline (e.g., published medical dictionaries), one or more training manuals/guidebooks/textbooks available in the given discipline (e.g., published medical training manuals/guidebooks/textbooks), encyclopedias, or information from one or more experts in the discipline (e.g., clinicians, physicians, etc.). The computing device(s) within the given shard may review the plurality of snippets within the respective training group to determine whether any of the words/series of words in the plurality of snippets represent expanded forms present within the one or more dictionaries (e.g., search for “patient” within the plurality of snippets using a dictionary that includes “pt: patient”). For each of the expanded forms in the training group that is present within the one or more dictionaries, the one or more computing devices in the shard may determine how many times that expanded form occurs within the training group of snippets. Based on the number of times the expanded form occurs within the training group of snippets, the one or more computing devices may then calculate the inclusion value for that respected expanded form. For example, the inclusion value may be calculated as:

$$p = \frac{1}{{( n + 1 )}^{α}}$$

where p is the inclusion value, n is the number of occurrences of the expanded form within the given training group, and α is a hyper-parameter of the training procedure. In some embodiments, (n+1)^{α} may be referred to as a “frequency value.” Further, in some embodiments, the method 300 may be repeated multiple times with different values of α (e.g., repeated 10,000 times with 10,000 pseudo-randomly selected values of α). The results of the multiple repetitions of the method 300 may then be compared (e.g., by comparing results from an inference phase that makes use of a machine-learned model trained using the training set generated according to the method 300) to determine which value of α ultimately yields the most appropriate training set for a given context. For example, the criterion for which value of α yields the most appropriate training set may be based on deriving statistical properties of the training set (e.g., the incidence of succinct representations) and selecting the value of α that gives the same values for those statistical properties as corresponding statistical properties determined for a corpus of collected medical notes (e.g., generated by physicians and describing corresponding real human subjects). Alternatively, a value for α may be chosen that is found to maximize one of the metrics explained below with reference to FIG. 6A (e.g., the “expansion accuracy” or “total accuracy”).

At step 310, upon calculating the plurality of inclusion values for the respective training groups, the method 300 may include each of the shards (e.g., each of the one or more computing devices in the shards) determining, for each of the snippets within the training group, whether to ultimately include the snippet within the resulting training set (e.g., for use in training the machine-learned model) based on the snippet within the training group that has the highest inclusion value (e.g., highest inclusion value p, defined above, among all the inclusion values in the training group). For example, each of the snippets in the training group may be probabilistically sampled (i.e., used to generate the resulting training set) with a probability equal pₘₐₓ (i.e., the highest inclusion value p among all inclusion values p in the training group). By weighting the inclusion of snippets within the resulting training set in this way, an effective upsampling of the less common expanded forms (e.g., less common medical terms) may occur while an effective downsampling of the more common expanded forms (e.g., more common medical terms). Such upsampling/downsampling may result in a training set having an improved effectiveness (e.g., improved effectiveness when used to train a machine-learned model), for example.

At step 312, upon each of the shards selecting which of the snippets to include within the training group, the method 300 may include performing a reserve substitution on the one or more snippets to be included within the training group using a reverse substitution probability. For example, a probabilistic determination (e.g., using a predetermined probability) of whether to perform reverse substitution (i.e., replacement of an expanded form with a succinct representation) may be made for each expanded form identified within the snippet (e.g., identified based on the associated dictionary described above). For instance, 95% (or some other percentage based on the predetermined probability, such as 90%, 85%, 80%, etc.) of the expanded forms across all snippets may be replaced by their corresponding succinct representations (e.g., based on the corresponding succinct representations defined within the dictionary, as described above). As illustrated in FIG. 3, this may result in a set of snippets having succinct representations for each of the shards. It is understood that, in some embodiments, reverse substitution may be applied to 100% of the expanded forms (i.e., a predetermined probability of 100% is used for the reverse substitution of step 312). It is also understood that, in some embodiments, steps 308 and 310 could be performed after step 312 (e.g., the reverse substitution of one or more expanded forms within a given snippet may occur before a determination of whether to ultimately include the reverse-substituted snippet into the training set is made).

At step 314, upon performing the probabilistic reverse substitution of step 312, the various snippets in each shard may be combined to form a resulting training set. For example, the snippets in each of the training sets may come in pairs (e.g., an original snippet taken from the publicly available web data and an associated snippet that results from reverse substitution). The resulting training set may therefore include a list of input snippets (e.g., snippets that include succinct representations of various terms) and a corresponding list of output snippets (e.g., snippets that should result when properly expanding the succinct representations into expanded forms). As such, this training set can be used to train a machine- learned model to expand snippets of text (e.g., expand a snippet of text such that some or all of the succinct representations are replaced by expanded representations).

As described herein, while the method 300 of FIG. 3 can be used to generate a training set of snippets within the medical context (e.g., accounting for abbreviations, acronyms, etc. within clinical notes), the method 300 of FIG. 3 is broadly applicable. As such, the method 300 of FIG. 3 can be used to generate training sets for a number of applications using publicly available web data.

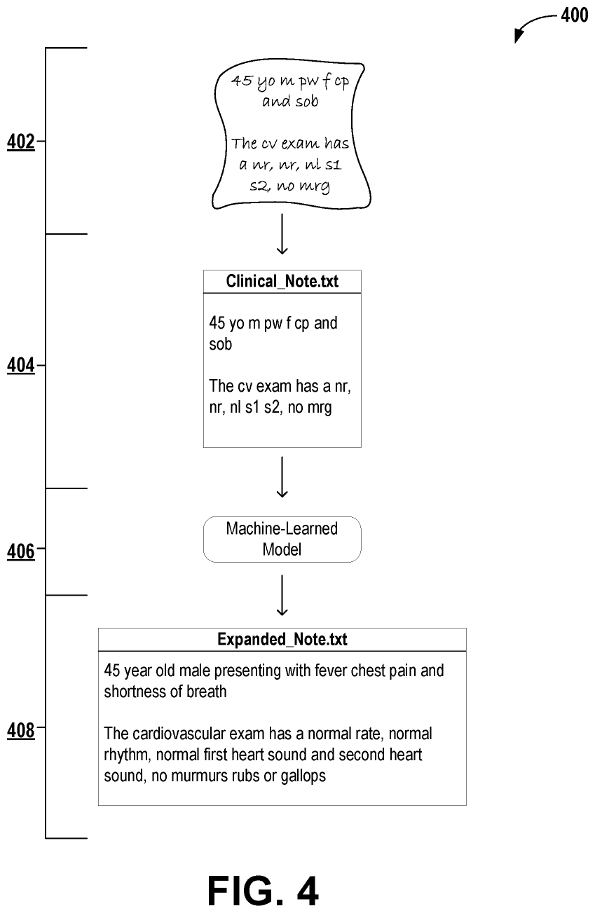

FIG. 4 is an illustration of a method 400 of expanding text within a clinical note (e.g., using a machine-learned model), according to example embodiments. For example, FIG. 4 is an illustration of a method 400 of expanding a clinical note using a machine-learned model trained using a training set generated according to the method 300 of FIG. 3. The method 400 can be performed in the inference phase of diagram 100 of FIG. 1 (e.g., by the computing device 200 of FIG. 2). The clinical note may include one or more snippets of text (e.g., which include one or more succinct representations of one or more medical terms). In some embodiments, the method 400 may be performed using one or more computing devices (e.g., the computing device 200 shown and described with respect to FIG. 2). While the method 400 of FIG. 4 is shown and described relative to a clinical note, it is understood that this is provided solely as an example and that other examples are also contemplated herein. For example, similar techniques could be used within the medical context (e.g., expanding a discharge summary prepared by a physician) or outside the medical context (e.g., expanding terminology in a corporate report that uses internal code names or jargon).

At step 402, the method 400 may include receiving a medical note. As illustrated, the medical note may be handwritten (e.g., on a prescription notepad or other clinical notepad) by a clinician, physician, nurse, physician's assistant, etc. Alternatively, the medical note may have been entered into a software (e.g., and stored within a memory, such as a hard drive or a cloud storage device). For example, a physician may enter a clinical note (e.g., using a keyboard, stylus, etc.) in an electronic form (e.g., a .txt file, a .pdf file, a .png file, etc.) that is then associated with a patient and stored (e.g., within an electronic health record). Receiving the medical note may include capturing an image (e.g., using a scanner or a camera) of a handwritten note. Alternatively, receiving the medical note may include retrieving the medical note from a repository (e.g., a cloud storage associated with an electronic health record for a patient). In some embodiments, receiving the medical note may include entering credentials (e.g., username and password) in order to access the medical note.

At step 404, the method 400 may include transforming the medical note from step 402 into a usable form. For example, in embodiments where the note of step 402 was handwritten and an image of the handwritten note was captured by a camera to obtain an electronic copy of the medical note, step 404 may include performing optical character recognition (OCR) to transform the captured image into a processible form (e.g., from a .png file to a .txt file). It is understood that, in some embodiments, step 404 may not occur. For example, if the medical note that was received in step 402 is already in an appropriate electronic form (e.g., based on the way it was stored within the electronic health record from which it was retrieved), a transformation of the medical note to a different form may not be necessary.

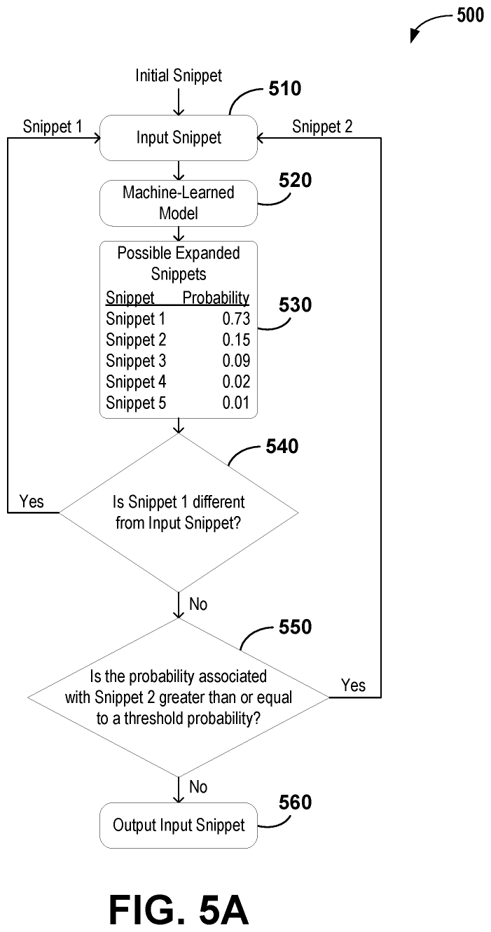

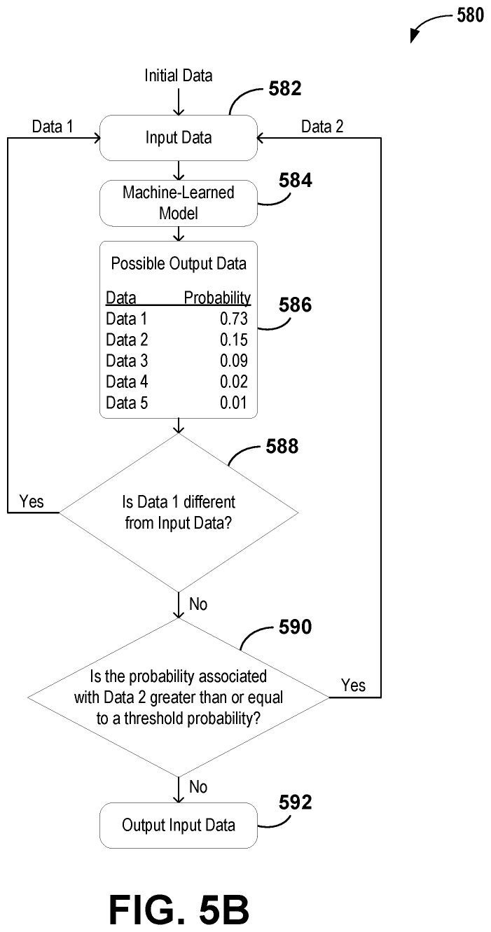

At step 406, the method 400 may include applying a machine-learned model to the electronic form of the medical note of step 404. For example, step 406 may include applying a machine-learned model (e.g., an ANN) that was trained using a training set generated according to the method 300 shown and described with reference to FIG. 3. Applying the machine-learned model in step 406 may include performing one or more inferences using the machine-learned model. Further, performing the one or more inferences may include performing the method 500 shown and described below with reference to FIG. 5A.

At step 408, the method 400 may include outputting the result of the application of the machine-learned model in step 406. The output may include one or more snippets of text (e.g., corresponding to the one or more snippets contained within the original clinical note of step 402). Further, each of the one or more snippets of text in the result in step 408 may include one or more expanded forms of one or more succinct representations contained within the clinical note of step 402. In some embodiments, outputting the result of the application of the machine-learned model may include generating a .txt file, a .pdf file, etc. that represents the output text. Further, outputting the result of the application may include storing a generated file (e.g., within a hard drive, a cloud storage, etc.) and/or associating the generated file with a given patient, clinician, physician, hospital, clinic, etc. (e.g., within an electronic health record). As only one example, in the method 400 of FIG. 4, the clinical note containing succinct representations reads: “45 yo m pw f cp and sob” and “The cv exam has a nr, nr, nl s1 s2, no mrg.” This clinical note has been transcribed/expanded into a .txt file that includes the following: “45 year old male presenting with fever chest pain and shortness of breath” and “The cardiovascular exam has a normal rate, normal rhythm, normal first heart sound and second heart sound, no murmurs rubs or gallops.”

FIG. 5A is an illustration of an iterative, elicitive inference method 500 (e.g., performed using a machine-learned model), according to example embodiments. The method 500 may be performed by a computing device (e.g., the computing device 200 shown and described with reference to FIG. 2). Further, the method 500 may be performed to expand succinct representations (e.g., succinct representations of medical terms) contained in one or more snippets of text (e.g., snippets of text taken from a clinical note, as illustrated in FIG. 4). Expanding such succinct representations may make the one or more snippets of text more easily understood, consistent, and/or unambiguous (e.g., to a patient, to another physician, within a medical record, etc.).

At step 510, the method 500 may include receiving an input snippet of text. The input snippet of text may be a snippet from a previous iteration of the method 500 (e.g., as illustrated by Snippet 1 and Snippet 2 in FIG. 5A). However, in the first iteration of the method 500, the input snippet of text may be an initial snippet (e.g., provided when initiating the method 500), as illustrated in FIG. 5A. For example, the initial snippet may be a snippet of text (e.g., in its original form) retrieved from a clinical note (e.g., as illustrated in FIG. 4), whereas intermediate snippets (e.g., Snippet 1 and/or Snippet 2 in FIG. 5A) may be partially expanded snippets (e.g., may have one or more of the succinct representations in the intermediate snippet already expanded into an expanded form).

At step 520, the method 500 may process the snippet of text received at step 510 using a machine-learned model. For example, step 520 may include inputting the snippet of text into a machine-learned model trained using a training set generated according to the method 300 illustrated in FIG. 3.

At step 530, the method 500 may include producing a list of possible expanded snippets of text. The list may be produced by the machine-learned model applied in step 520, for example. In some embodiments, the list may be produced by performing a beam search using the machine-learned model. The list may include possible expanded snippets along with corresponding probabilities that the given expanded snippet is the correct expanded form of the snippet. The corresponding probabilities may be generated based on the machine-learned model, for example. In some embodiments, for instance, the machine-learning model may include a classifier trained to output a set of possible expanded snippets, each with an associated probability of being correct. Additionally or alternatively, the machine-learning model could include an encoder-decoder Text-to-Text Transfer Transformers (i.e., T5), such as a T5 80B trained on a web corpus using masked language modeling (MLM) loss. Example T5s are described in more detail in “Exploring the limits of transfer learning with a unified text-to-text transformer;” Colin Raffel, et al.; J. Mach. Learn. Res. 21.140 1-67 (2020). Upon generating the list of possible expanded snippets of text, the list may be ordered from highest corresponding probability to lowest corresponding probability (e.g., as illustrated in FIG. 5A). While only five possible expanded snippets of text are illustrated in FIG. 5A, it is understood that this is provided solely as an example and that other embodiments are also possible and are contemplated herein. For example, one, two, three, four, six, seven, eight, nine, ten, etc. possible expanded snippets of text might be generated (e.g., by a beam search performed using the machine-learned model) in various embodiments. Further, the corresponding probabilities illustrated in FIG. 5A are also provided solely as examples. Other possible corresponding probabilities are also possible and are contemplated herein.

At step 540, the method 500 may include determining whether the possible expanded snippet of text with the highest corresponding probability in the list (e.g., the list generated at step 530) is different from the input snippet (e.g., the snippet input into the method at step 510 for the current iteration of the method 500). This may include performing a character-by-character comparison of the input snippet to the possible expanded snippet of text (e.g., Snippet 1 in FIG. 5A) to determine whether the two are the same. If the two snippets are different from one another, the method 500 may proceed to step 510 by providing the possible expanded snippet of text with the highest corresponding probability (e.g., Snippet 1 in FIG. 5A) as an input to step 510. If the two snippets are the same as one another, the method 500 may proceed to step 550.

At step 550, the method 500 may include determining whether the second highest corresponding probability in the list (e.g., the list generated at step 530) has a value that is greater than or equal to a threshold probability. In other words, step 550 may include comparing the probability corresponding to the second most likely possible expanded snippet of text (e.g., Snippet 2 illustrated in FIG. 5A) to a threshold probability value. If the second highest corresponding probability in the list is greater than or equal to the threshold probability, the method 500 may proceed to step 510 by providing the possible expanded snippet of text with the second highest corresponding probability (e.g., Snippet 2 in FIG. 5A) as an input to step 510. If the second highest corresponding probability in the list is less than the threshold probability, the method 500 may proceed to step 560.

The threshold probability value may be a hyper-parameter associated with the machine-learned model, for example. Further, it may be determined by empirically testing a plurality of different possible threshold probability values and/or based on feedback from industry professionals (e.g., one or more physicians). As such, in various embodiments, the threshold probability value may have different values. For example, the threshold probability value may be 0.40, 0.35, 0.30, 0.25, 0.20, 0.15, 0.10, 0.05, 0.01, 0.005, 0.001, 0.0005, 0.0001, 0.00005, 0.00001, etc.

At step 560, the method 500 may include outputting the input snippet (e.g., the snippet input into the method at step 510 for the current iteration of the method 500). Outputting the input snippet may include displaying the input snippet (e.g., on a display such as a monitor), saving the input snippet (e.g., in an electronic format), providing the snippet as an input to another method (e.g., a method also being executed by the computing device performing the method 500 or being executed by a different computing device), combining the input snippet with another snippet of text (e.g., another snippet previously generated using the method 500), etc. According to the method 500, reaching step 560 on the current iteration of the method 500 indicates that the possible expanded snippet with the highest corresponding probability (e.g., Snippet 1 in FIG. 5A) is the same as the input snippet and that the second highest corresponding probability (e.g., the probability corresponding to Snippet 2 in FIG. 5A) has a value that is less than the threshold probability. Hence, it is possible to determine that the input snippet should be output as the final expanded snippet (e.g., representing the most appropriate expanded form producible by the machine-learned model given the current hyper-parameter(s)).

While the illustrated method 500 of FIG. 5A includes computing and comparing probabilities (e.g., at step 530 and step 550), it is understood that the use of probabilities is provided solely as an example and that other embodiments are also possible and contemplated herein. In general, the method 500 of FIG. 5A may include computing scores, comparing scores to one another, and/or comparing scores to one or more threshold scores. While scores/threshold scores may include one or more probability values, additional or alternative types of scores are also possible.

Further, while the method 500 shown and described with reference to FIG. 5A is used to expand snippets of text, it is understood that such an elicitive, iterative inference method is broadly applicable and could be applied to other types of data/machine-learned models (e.g., machine-learned models that were not trained using training data generated according to the method 300 shown and described with reference to FIG. 3). A genericized version of the method 500 of FIG. 5A is illustrated in FIG. 5B. The method 580 of FIG. 5B (similar to the method 500 of FIG. 5A) may include receiving input data at step 582, processing the input data using a machine-learned model at step 584, producing a list of possible output data with associated probabilities at step 586, determining whether the possible output data with the highest corresponding probability in the list is different from the input data at step 588, determining whether the second highest corresponding probability in the list has a value that is greater than or equal to a threshold probability at step 590, and outputting the input data at step 592. The method 580 of elicitive, iterative inference may provide a generic improvement to computer-related technology by producing enhanced results (e.g., results having higher accuracy) compared to alternative inference methods.

### III. Experimental Results

FIGS. 6A-6D illustrate experimental results of example embodiments disclosed herein when compared against alternative techniques. Some of the alternative techniques described may include applying different inference types using a machine-learned model trained according to a training set as described herein (e.g., the training set generated according to the method 300 of FIG. 3). Additionally, FIGS. 6A-6D may be used to illustrate performance using the machine-learned model as described herein compared with groups individuals of varying experience levels (e.g., comparing expanded snippets generated by laypersons to expanded snippets generated by medical students to expanded snippets generated using a machine-learned model according to embodiments described herein).

To evaluate the techniques described herein, three test datasets were used. It is understood that other datasets may also be used with techniques described herein (e.g., including non-medical/non-clinical datasets); the three datasets below were simply used to perform a comparative analysis of example embodiments. The three datasets include:

CASI dataset—The CASI dataset was generated based on “Clinical Abbreviation Sense Inventory;” Sungrim Moon, et al.; retrieved from the University of Minnesota Digital Conservancy (2012). As described by the authors, the CASI dataset “contains 440 common abbreviations and acronyms from a corpus of 604,944 dictated clinical notes (2004-8) containing discharge summaries, operative reports, consultation notes, and admission notes.” Further, in the CASI dataset used herein, examples from the publication described above that included abbreviations where the actual abbreviation appears in some modified form in the original snippet (e.g., pm appears as “p.m.”) and snippets that included more than 100 tokens were removed. This resulted in the CASI dataset, as used herein, having 21,514 snippets of text, which included 64 unique abbreviations and 122 unique abbreviation-expansion pairs.

MIMIC dataset—The MIMIC dataset includes 5,005 snippets containing 2,579 unique abbreviation-expansion pairs across 12,206 labeled abbreviations. The dataset was generated by adapting de-identified discharge summaries from an intensive care unit at the Beth Israel Deaconess Medical Center in Massachusetts (MIMIC III; “MIMIC-III, A Freely Accessible Critical Care Database;” A. E. W. Johnson, et al.; Sci Data 3, 160035 (2016)). This dataset was generated based on 59,652 discharge notes by splitting into sentences using a delimiter of a period followed by the space character. Then, only snippets between 40 and 200 characters that did not include brackets were kept, resulting in 3,092,981 snippets. This was further reduced by converting to lowercase and removing duplicates (resulting in 2,377,834 snippets). 95.4% of the remaining snippets included at least one expanded form included in the dictionary used. Each of the expanded forms in the remaining snippets were then replaced with the succinct representation at a rate of 10,000 divided by the total number of occurrences of the given expanded form (in an attempt to maintain only about 10,000 instances of any given succinct representation). If a given expanded form had fewer than 10,000 occurrences, all occurrences of a given expanded form were replaced by a corresponding succinct representation. Finally, the converted snippets were sampled if the snippet included at least one succinct representation that had not been previously included in other snippets at least three times. This resulted in 5,005 snippets containing 2,579 unique abbreviation-expansion pairs across 12,206 labeled abbreviations.

Synthetic snippets dataset—This dataset of snippets of clinical text was generated by asking senior medical students, residents, and attending physicians to generate sentences that contained medical abbreviations that were randomly selected from previously published abbreviations in clinical notes and sign-out sheets. Since many succinct representations can be ambiguous (e.g., “af” for “atrial fibrillation,” as well as “afebrile”), snippets for each distinct expanded representation were generated. This ultimately resulted in 302 synthetic snippets. Further, the clinicians generated a key that indicated how each abbreviation was meant to be expanded.

In evaluating the techniques described herein against alternative techniques a variety of metrics may be used. For example, detection recall, detection precision, expansion accuracy, and/or total accuracy may be considered. Such metrics are defined in the following ways:

Detection recall—The percent of actual succinct representations (e.g., actual abbreviations) of snippets of text that were expanded using the machine-learned model. A higher detection recall percentage corresponds to a higher likelihood that the machine-learned model correctly detects that a given word or phrase contains a succinct representation that should be expanded.

Detection precision—The percent of expansions made by the machine-learned model that actually contained succinct representations (e.g., abbreviations). A higher detection precision corresponds to a lower likelihood that the machine-learned model incorrectly expands succinct representations that were not meant as succinct representations. This may be important as certain combinations of characters (e.g., the letters “it”) can function both as succinct representations (e.g., abbreviations for “iliotibial” or “intrathecal”) or as a word, depending on context.

Expansion accuracy—The percent of expansions of correctly detected succinct representations (e.g., abbreviations) made using the machine-learned model that had the same clinical meaning as the original text (e.g., as the original succinct representation). Because some long-forms are clinically equivalent, the expanded form could be distinct from the target label (e.g., the “proper” expansion) but still be clinically equivalent (e.g. “CCU” could equivalently refer to “cardiac care unit” or “coronary care unit”). In order to determine whether two strings were clinically equivalent, an attending physician in internal medicine adjudicated each snippet in the test set used to generate FIGS. 6A and 6D. For the CASI dataset and MIMIC dataset (e.g., shown and described in FIGS. 6B and 6C), however, the adjudication for each snippet was not feasible. As such, the expansion accuracy for the CASI dataset and MIMIC dataset is reported against the single intended label (e.g., the “proper” expansion) or strings marked as equivalent in a general sense. Hence, the metrics shown and described for the example embodiments in FIGS. 6B and 6C are inherently conservative.

Total accuracy—The percent of succinct representations that were identified and replaced with the originally intended or clinically equivalent expansion. The total accuracy can be calculated, for example, by multiplying the detection recall (number of correctly detected succinct representations out of all succinct representations) with the expansion accuracy (the number of correctly expanded succinct representations out of correctly detected succinct representations).

FIG. 6A is a table showing the four metrics described above when applying a machine-learned model trained using a training set generated using publicly available web data (e.g., generated according to the method 300 shown and described with reference to FIG. 3). The results illustrated in FIG. 6A were generated using the synthetic snippets dataset described above. Further, the results illustrated in FIG. 6A were generated by applying the machine-learned model using three different types of inference: single model inference, iterative inference, and elicitive and iterative inference (e.g., example embodiments described herein, such as above with reference to the method 500 of FIG. 5A).

Single model inference may refer to performing an inference that simply involves applying the machine-learned model and producing an initial output. For example, single model inference may refer to using a machine-learned model that is trained using the training set generated according to the method 300 of FIG. 3, receiving an input snippet, determining whether an output snippet should be produced (e.g., determining if there are any succinct representations in a given snippet), and expanding the succinct representations in the given snippet, if necessary, to produce an output snippet using the machine-learned model. This may be repeated for all possible input snippets in the synthetic snippets dataset, and the resulting output snippets may then be compared to the actual, correct expanded snippets in the synthetic snippets dataset to determine the metrics of FIG. 6A corresponding to the “single model inference.”

Iterative inference may refer to performing a repeated inference using the machine-learned model to produce a final output. For example, the machine-learned model may determine whether to expand a given snippet and then generate an intermediate expansion. If the intermediate expansion is different from the input snippet, the machine-learned model may repeat the expansion process using the intermediate expansion as an input. This may be repeated until the intermediate expansion is the same as the input snippet. If the intermediate expansion is the same as the input snippet, the input snippet may be output as the final output snippet. This may be repeated for all possible input snippets in the synthetic snippets dataset, and the resulting output snippets may then be compared to the actual, correct expanded snippets in the synthetic snippets dataset to determine the metrics of FIG. 6A corresponding to the “iterative inference.”

Finally, iterative and elicitive inference may refer to example embodiments described herein that perform repeated inference and not only compare intermediate expansions to the input snippet, but also check the score (e.g., probability) associated with a next-most likely expansion. For example, the method 500 described with reference to FIG. 5A may be described as “iterative and elicitive inference.” The process of iterative elicitive inference (e.g., the method 500 of FIG. 5A) may be repeated for all possible input snippets in the synthetic snippets dataset, and the resulting output snippets may then be compared to the actual, correct expanded snippets in the synthetic snippets dataset to determine the metrics of FIG. 6A corresponding to the “iterative and elicitive inference.”

As illustrated in FIG. 6A, the detection recall and the total accuracy of the iterative and elicitive inference are higher than either the single model inference or the iterative inference. Further, the detection precision for all three types of inference were the same. The iterative and elicitive inference only has slightly lower values than the single model inference and the iterative inference for the expansion accuracy.

FIG. 6B illustrates three of the four metrics described above (e.g., detection recall, expansion accuracy, and total accuracy) for example embodiments described herein (e.g., the method 500 of inference of FIG. 5A applied using a machine-learned model trained using a dataset generated according to the method 300 of FIG. 3) as applied to the CASI dataset. Specifically, FIG. 6B illustrates three of the four metrics described above for example embodiments described herein as applied to three subsets of the CASI dataset. For example, example embodiments have been applied to ambiguous snippets within the CASI dataset (i.e., those snippets that include succinct representations that can be expanded in more than one way per the dictionary used herein), non-ambiguous snippets within the CASI dataset (i.e., those snippets that include succinct representations that can only be expanded in a single way per the dictionary used herein), and all the snippets within the CASI dataset. As illustrated, the three metrics for each of the subsets demonstrate substantial performance of the machine-learned model described herein (e.g., each of the detection recall, expansion accuracy, and total accuracy in each of the three subsets is over 0.910).

FIG. 6C illustrates three of the four metrics described above (e.g., detection recall, expansion accuracy, and total accuracy) for example embodiments described herein (e.g., the method 500 of inference of FIG. 5A applied using a machine-learned model trained using a dataset generated according to the method 300 of FIG. 3) as applied to the MIMIC dataset. Specifically, FIG. 6C illustrates three of the four metrics described above for example embodiments described herein as applied to three subsets of the MIMIC dataset. For example, example embodiments have been applied to ambiguous instances within the MIMIC dataset (i.e., those succinct representations that can be expanded in more than one way per the dictionary used herein), non-ambiguous instances within the MIMIC dataset (i.e., those succinct representations that can only be expanded in a single way per the dictionary used herein), and all instances within the MIMIC dataset. The results have been further delineated based on whether the succinct representations were considered “common” (i.e., there are at least 500 instances of the given succinct representation in the MIMIC dataset), “uncommon” (i.e., there are at least 10 instances, but fewer than 500 instances, of the given succinct representation in the MIMIC dataset), or “rare” (i.e., there are fewer than 10 instances of the given succinct representation in the MIMIC dataset). As illustrated, the three metrics for each of the possible subsets demonstrate substantial performance of the machine-learned model described herein (e.g., each of the detection recall, expansion accuracy, and total accuracy in each of the possible subsets is over 0.950).

The task of taking a snippet and outputting the equivalent snippet with the succinct representations expanded can be considered a type of translation. In order to evaluate how well the machine-learned models/inference techniques described herein perform this task, a basis of comparison to human performance may be helpful. In order to make such a comparison, a baseline of how well a human may perform the translation task described herein has been generated. In order to generate the baseline, thirty snippets from the synthetic snippets dataset, each with at least three succinct representations, were chosen. Individuals within four groups were asked to translate (i.e., expand the succinct representations within the thirty snippets). The four groups included: three laypeople attempting to perform the translation themselves, three different laypeople attempting to perform the translation with the use of an internet search engine (e.g., GOOGLE search engine), three medical students, and three physicians who were board certified in internal medicine (i.e., “attending physicians”). The latter two groups were not allowed to use internet searches to simulate a time-pressured clinical environment. The laypeople in both layperson groups were engineers who were competent in using internet searches. All groups were instructed to not expand succinct representations where they were not reasonably confident in the expanded form. The results of the attempted expansion by these four groups, as well as the results of the machine-learned models/inference techniques described herein applied to the same snippets, are presented in FIG. 6D.

As illustrated in FIG. 6D, the laypeople without internet search attained a total accuracy of 28.6%, with low performance largely driven by lack of knowledge regarding how to expand succinct representations (e.g., a detection recall of 34.9%). Access to internet search improved layperson detection recall to 82.9%, driving total accuracy up to 75.6%. Medical students and attending physicians performed with high total accuracies of 89.5% and 88.5%, respectively. Example embodiments as described herein, though, had the highest total accuracy of all groups of 99.2%, also having the highest detection recall (e.g., 100%). In this evaluation, part of this discrepancy was that attending physicians did not expand succinct representations that are commonly used as succinct representations (e.g. choosing not to expand “cm” to “centimeter”). As is illustrated in FIG. 6D, the machine-learned models and inference techniques described herein represent improvements to translation relative to human performance.

### IV. Example Processes

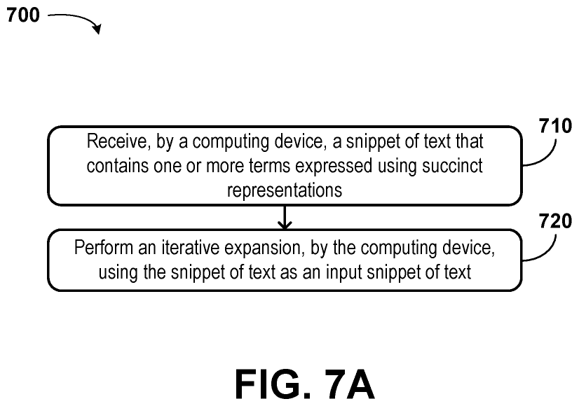

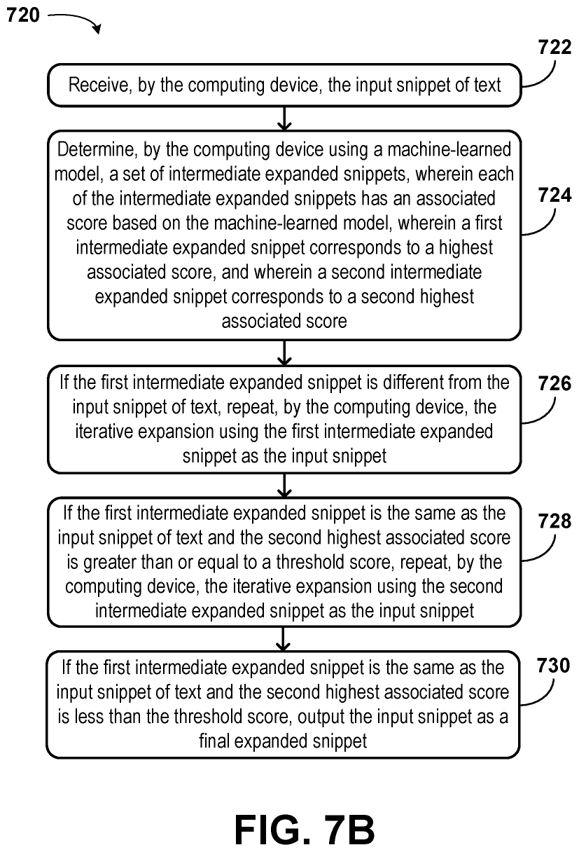

FIG. 7A is a flowchart diagram of a method 700, according to example embodiments. In some embodiments, the method 700 may be a computer-implemented method for expanding succinct representations (e.g., medical abbreviations originally contained in clinical note). In some embodiments, the method 700 may be performed by one or more processors of a system executing instructions stored in a non-transitory, computer-readable medium. The one or more processors may include one or more GPUs or one or more TPUs, for example.

At block 710, the method 700 may include receiving, by a computing device, a snippet of text that contains one or more terms expressed using succinct representations. In some embodiments of the method 700, the snippet of text may relate to a clinical note. Further, the one or more terms may correspond to medical terminology and/or the succinct representations may correspond to abbreviations of the medical terminology.

In some embodiments, the method 700 may also include receiving, by the computing device, an image of the clinical note. The clinical note may be handwritten, for example. Further, the method 700 may also include performing, by the computing device, an optical character recognition of the clinical note to determine the snippet of text.

In some embodiments, block 710 may also include retrieving, from an electronic health record stored within a server, the clinical note.

In some embodiments of the method 700, the succinct representations may correspond to internal code names used within a corporation.

At block 720, the method 700 may include performing an iterative expansion, by the computing device, using the snippet of text as an input snippet of text. FIG. 7B is a flow diagram illustrating example processes performed as part of block 720.

For example, at block 722, performing the iterative expansion may include receiving, by the computing device, the input snippet of text.

At block 724, performing the iterative expansion may include determining, by the computing device using a machine-learned model, a set of intermediate expanded snippets, wherein each of the intermediate expanded snippets has an associated score based on the machine-learned model, wherein a first intermediate expanded snippet corresponds to a highest associated score, and wherein a second intermediate expanded snippet corresponds to a second highest associated score. In some embodiments, block 724 may include performing a beam search. Further, in some embodiments, the machine-learned model may have been trained using reverse substitution. Additionally or alternatively, in some embodiments, the machine-learned model may have been trained using public website data retrieved using a webcrawler. Still further, the public website data may have been presented in a different form that the snippet of text. For example, the public website data may include website data relating to explanations of medical conditions and the snippet of text may have been retrieved from a clinical note.

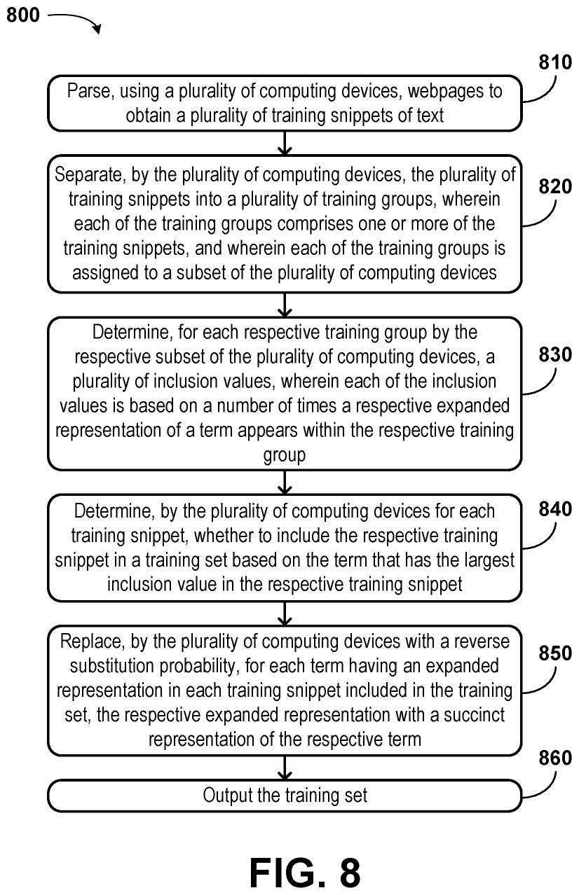

In some embodiments of the method 700, the machine-learned model may have been trained using an enhanced reverse substitution process (e.g., may have been trained using a process similar to the method 800 shown and described below with reference to FIG. 8). For example, the enhanced reverse substitution process may include parsing webpages to obtain a plurality of training snippets of text. The enhanced reverse substitution process may also include separating the plurality of training snippets into a plurality of training groups. Each of the training groups may include one or more of the training snippets. Additionally, the enhanced reverse substitution process may include determining, for each training group, a plurality of inclusion values. Each of the inclusion values may be based on a number of times a respective expanded representation of a term appears within the respective training group. For example, each of the plurality of inclusion values may be determined to be equal to an inverse of a frequency value (e.g., where the frequency value, p, is equal to the number of times the respective expanded representation of a term appears within the respective training group plus one to the power of a hyper-parameter α, i.e.,

$$p = \frac{1}{{( n + 1 )}^{α}} ) .$$

Further, the enhanced reverse substitution process may include determining, for each training snippet, whether to include the respective training snippet in a training set based on the term that has the largest inclusion value in the respective training snippet. In addition, the enhanced reverse substitution process may include replacing, with a reverse substitution probability (e.g., between 90% and 100%), for each term having an expanded representation in each training snippet included in the training set, the respective expanded representation with a succinct representation of the respective term. Yet further, in some embodiments of the method 700, the training snippets may include between one and three sentences of text. Even further, in some embodiments of the method 700, the machine-learned model may have been trained by applying the enhanced reverse substitution process multiple times for different values of a hyper-parameter a and comparing the resulting training sets. Even still further, in some embodiments of the method 700, the machine-learned model may have been trained by applying the enhanced reverse substitution process on a plurality of computing devices to generate a plurality of training sets and then selecting one of the training sets from among the plurality of training sets for use in training the machine-learned model.

At block 726, performing the iterative expansion may include, if the first intermediate expanded snippet is different from the input snippet of text, repeat, by the computing device, the iterative expansion using the first intermediate expanded snippet as the input snippet.

At block 728, performing the iterative expansion may include, if the first intermediate expanded snippet is the same as the input snippet of text and the second highest associated score is greater than or equal to a threshold score, repeat, by the computing device, the iterative expansion using the second intermediate expanded snippet as the input snippet. In some embodiments, the threshold score may be a hyper-parameter of the machine-learned model. Further, the threshold score may be empirically selected from among a variety of threshold probabilities based on a validated set of data that has been manually reviewed to determine which of the threshold probabilities yields a best result.

At block 730, performing the iterative expansion may include, if the first intermediate expanded snippet is the same as the input snippet of text and the second highest associated score is less than the threshold score, output the input snippet as a final expanded snippet.

In some embodiments, block 730 may include transmitting the input snippet as the final expanded snippet to a server for storage in an electronic health record.

In some embodiments, block 730 may include displaying the input snippet as the final expanded snippet on a display. The final expanded snippet may be usable by a physician for diagnosis or treatment.

FIG. 8 is a flowchart diagram of a method 800, according to example embodiments. In some embodiments, the method 800 may be performed to generate a training set that can be used to train a machine-learned model (e.g., a machine-learned model that is usable to expand abbreviations). In some embodiments, the method 800 may be performed by one or more processors of a system executing instructions stored in a non-transitory, computer-readable medium. The one or more processors may include one or more GPUs or one or more TPUs, for example.

At block 810, the method 800 may include parsing, using a plurality of computing devices, webpages to obtain a plurality of training snippets of text.

At block 820, the method 800 may include separating, by the plurality of computing devices, the plurality of training snippets into a plurality of training groups, wherein each of the training groups comprises one or more of the training snippets, and wherein each of the training groups is assigned to a subset of the plurality of computing devices.

At block 830, the method 800 may include determining, for each respective training group by the respective subset of the plurality of computing devices, a plurality of inclusion values, wherein each of the inclusion values is based on a number of times a respective expanded representation of a term appears within the respective training group.

At block 840, the method 800 may include determining, by the plurality of computing devices for each training snippet, whether to include the respective training snippet in a training set based on the term that has the largest inclusion value in the respective training snippet.

At block 850, the method 800 may include replacing, by the plurality of computing devices with a reverse substitution probability, for each term having an expanded representation in each training snippet included in the training set, the respective expanded representation with a succinct representation of the respective term.

At block 860, the method 800 includes outputting the training set.

### V. Conclusion

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1. A method comprising:
receiving, by a computing device, a snippet of text that contains one or more terms expressed using succinct representations; and
performing an iterative expansion, by the computing device, using the snippet of text as an input snippet of text, wherein the iterative expansion comprises:
receiving, by the computing device, the input snippet of text;
determining, by the computing device using a machine-learned model, a set of intermediate expanded snippets, wherein each of the intermediate expanded snippets has an associated score based on the machine-learned model, wherein a first intermediate expanded snippet corresponds to a highest associated score, and wherein a second intermediate expanded snippet corresponds to a second highest associated score;
if the first intermediate expanded snippet is different from the input snippet of text, repeating, by the computing device, the iterative expansion using the first intermediate expanded snippet as the input snippet;
if the first intermediate expanded snippet is the same as the input snippet of text and the second highest associated score is greater than or equal to a threshold score, repeating, by the computing device, the iterative expansion using the second intermediate expanded snippet as the input snippet; and
if the first intermediate expanded snippet is the same as the input snippet of text and the second highest associated score is less than the threshold score, outputting the input snippet as a final expanded snippet.

2. The method of claim 1, wherein the snippet of text relates to a clinical note describing a medical condition of a human subject, and wherein the one or more terms correspond to medical terminology and the succinct representations correspond to abbreviations of the medical terminology.

3. The method of claim 2, further comprising:
receiving, by the computing device, an image of the clinical note, wherein the clinical note is handwritten; and
performing, by the computing device, an optical character recognition of the clinical note to determine the snippet of text.

4. The method of claim 2, wherein receiving the snippet of text comprises retrieving, from an electronic health record stored within a server, the clinical note.

5. The method of claim 1, wherein the threshold score is a hyper-parameter of the machine-learned model.

6. The method of claim 5, wherein the threshold score is empirically selected from among a variety of threshold scores based on a validated set of data that has been manually reviewed to determine which of the threshold probabilities yields a best result.

7. The method of claim 1, wherein outputting the input snippet as the final expanded snippet comprises transmitting the input snippet as the final expanded snippet to a server for storage in an electronic health record.

8. The method of claim 1, wherein outputting the input snippet as the final expanded snippet comprises displaying the input snippet as the final expanded snippet on a display, and wherein the final expanded snippet is usable by a physician for diagnosis or treatment.

9. The method of claim 1, wherein determining, by the computing device using the machine-learned model, the set of intermediate expanded snippets comprises performing a beam search.

10. The method of claim 1, wherein the succinct representations correspond to internal code names used within a corporation.

11. The method of claim 1, wherein the machine-learned model was trained using reverse substitution.

12. The method of claim 1, wherein the machine-learned model is trained using public website data retrieved using a webcrawler.

13. The method of claim 12, wherein the public website data is presented in a different form than the snippet of text.

14. The method of claim 13, wherein the public website data comprises website data relating to explanations of medical conditions, and wherein the snippet of text is retrieved from a clinical note.

15. The method of claim 12, wherein the machine-learned model was trained using an enhanced reverse substitution process, and wherein the enhanced reverse substitution process comprises:
parsing webpages to obtain a plurality of training snippets of text;
separating the plurality of training snippets into a plurality of training groups, wherein each of the training groups comprises one or more of the training snippets;
determining, for each training group, a plurality of inclusion values, wherein each of the inclusion values is based on a number of times a respective expanded representation of a term appears within the respective training group;
determining, for each training snippet, whether to include the respective training snippet in a training set based on the term that has the largest inclusion value in the respective training snippet; and
replacing, with a reverse substitution probability, for each term having an expanded representation in each training snippet included in the training set, the respective expanded representation with a succinct representation of the respective term.

16. The method of claim 15, wherein the training snippets comprise between one and three sentences of text.

17. The method of claim 15, wherein the reverse substitution probability has a value between 90% and 100%.

18. The method of claim 15, wherein each of the plurality of inclusion values is determined to be equal to an inverse of a frequency value, and wherein the frequency value is equal to the number of times the respective expanded representation of a term appears within the respective training group plus one and to the power of a hyper-parameter α.

19. The method of claim 18, wherein the machine-learned model was trained by applying the enhanced reverse substitution process multiple times for different values of the hyper-parameter α and comparing the resulting training sets.

20. The method of claim 15, wherein the machine-learned model was trained by:
applying the enhanced reverse substitution process on a plurality of computing devices to generate a plurality of training sets; and
selecting one of the training sets from among the plurality of training sets for use in training the machine-learned model.

21. A method comprising:
parsing, using a plurality of computing devices, webpages to obtain a plurality of training snippets of text;
separating, by the plurality of computing devices, the plurality of training snippets into a plurality of training groups, wherein each of the training groups comprises one or more of the training snippets, and wherein each of the training groups is assigned to a subset of the plurality of computing devices;
determining, for each respective training group by the respective subset of the plurality of computing devices, a plurality of inclusion values, wherein each of the inclusion values is based on a number of times a respective expanded representation of a term appears within the respective training group;
determining, by the plurality of computing devices for each training snippet, whether to include the respective training snippet in a training set based on the term that has the largest inclusion value in the respective training snippet;
replacing, by the plurality of computing devices with a reverse substitution probability, for each term having an expanded representation in each training snippet included in the training set, the respective expanded representation with a succinct representation of the respective term; and
outputting the training set.

22. A non-transitory, computer-readable medium having instructions stored therein, wherein the instructions, when executed by a processor, perform a method comprising:
receiving a snippet of text that contains one or more terms expressed using succinct representations; and
performing an iterative expansion using the snippet of text as an input snippet of text, wherein the iterative expansion comprises:
receiving the input snippet of text;
determining, using a machine-learned model, a set of intermediate expanded snippets, wherein each of the intermediate expanded snippets has an associated score based on the machine-learned model, wherein a first intermediate expanded snippet corresponds to a highest associated score, and wherein a second intermediate expanded snippet corresponds to a second highest associated score;
if the first intermediate expanded snippet is different from the input snippet of text and the highest associated score is greater than a threshold score, repeating the iterative expansion using the first intermediate expanded snippet as the input snippet;
if the first intermediate expanded snippet is the same as the input snippet of text and the second highest associated score is greater than the threshold score, repeating the iterative expansion using the second intermediate expanded snippet as the input snippet; and
if the first intermediate expanded snippet is the same as the input snippet of text and the second highest associated score is less than the threshold score, outputting the input snippet as a final expanded snippet.
